(19) United States
(12) Patent Application Publication
(10) Pub. No.: US 2006/0122601 A1
(43) Pub. Date: Jun. 8, 2006

(54) Intramedullary nail

(76) Inventor: Vineet Dev Tandon, Sale (GB)

Correspondence Address: ARVIN F. LIESTER;2515 LASALLE STREET, COLORADO SPRINGS, CO, 80909, US

(21) Appl. No.: 11/339,555

(22) Filed: Jan. 26, 2006

Related U.S. Application Data
(63) Continuation-in-part of application No. PCT/GB04/03300, filed on Jul. 30, 2004.

(30) Foreign Application Priority Data
Jul. 31, 2003 (GB) 0317921.5

(51) Int. Cl. A61B 17/56

(52) U.S. Cl. 606/64

(57) ABSTRACT

Within the cavity (12) of an intramedullary nail, means such as a channel element (16) is mounted whereby a leading end (26) of a flexible elongate wire (22) is guidable from insertion into a proximal lateral aperture (20) to emerge from a distal lateral aperture (30), with the trailing end of the wire (22) still projecting from the proximal aperture (20). In this way when the nail is inserted into the medulla of a bone, across a fracture, it can be secured to the bone near both its ends. Two guide means, such as two channels (31, 33) of a channel element (16), with deflected guide wall portions (34, 38), or two conduits (FIGS. 8 to 13) guiding respective fixing wires between respective proximal and distal apertures, may usefully be provided.

### FIELD OF INVENTION

The present invention relates to an intramedullary nail as used for the repair of bones with fractures.

An intramedullary nail, typically consisting of a bio-acceptable metal such as titanium or a stainless steel alloy is used to support a fractured bone by insertion into the medulla of the bone. Such a nail may be inserted directly into the medulla, or if the medulla is too narrow, or a larger diameter nail is required, the medulla may first of all be reamed.

### BACKGROUND ART

There are two types of intramedullary nail: those which are solid for the most part and those which are hollow, or at least have a bore extending, generally axially, from one end to the other. The latter are termed reamed nails and they are usually inserted over a preliminary guide wire, which is inserted into the medulla, across the fracture, and is removed later, once the nail is in place.

Typically, the nail is inserted across the fracture under x-ray control, with use of an image intensifier.

After insertion of the nail into the cavity, it is preferable to secure the nail to the bone on either side of the fracture. This is typically done by inserting screws or bolts through the bone and through apertures in the nail near the proximal and distal ends of the nail. Securing the nail near its proximal end is not usually problematic as lateral holes can be drilled in the bone quite accurately by use of a suitable jig. However, drilling apertures near the distal end of the bone is problematic. The path of the nail is often slightly distorted as it passes down the medulla and across the fracture and only a minor degree of angulation or rotation of the nail makes the use of a jig inaccurate such that it will not align the hole for the screw or bolt accurately with the aperture near the distal end of the nail. To achieve the accuracy required for alignment with the one or more apertures in the nail, for placement of screws or other securing devices at the end of the nail remote from the site of insertion (distal end), it is necessary to take a number of X-rays, which is a time consuming procedure that can add significant time to an operation.

To deal with this problem various proposals have been made in respect of reamed nails, for example, as disclosed in U.S. Pat. No. 2,998,007, U.S. Pat. No. 6,309,392, EP-A-1155661 and EP-A-0517435, for inserting fixing wires into the hollow cavity of the nail from the proximal end and providing means for guiding tips of these wires out of lateral apertures near to the distal end of the nail to pierce the bone and thereby secure the nail. On the whole, these have not proved satisfactory. Where a wire of bent configuration is proposed, the leading end or tip may not be reliably directed out of the lateral aperture near the distal end of the nail. The proximal end of the nail should preferably still be secured to the bone in some way, but insertion of a transverse screw or bolt through the bone and through the aligned apertures near the proximal end of the bone in the conventional manner may be hindered or prevented by the presence of the fixing wire or wires which may still project to that level in the cavity.

### OBJECTS OF INVENTION

It is an object of the present invention to obviate or mitigate disadvantages with known nails, particularly those as previously described. It is also an object to provide means of securing intramedullary nails both quickly and accurately thereby improving on known devices and techniques.

### SUMMARY OF INVENTION

According to a first aspect of the present invention an intramedullary nail is provided comprising an elongate rod having a first end, a second end and an outer surface, a proximal lateral aperture in a region of the outer surface in the vicinity of the first end, a distal lateral aperture in a region of the outer surface in the vicinity of the second end, and a cavity through at least a portion of the rod, and means within the cavity whereby a flexible elongate wire having a leading end and a trailing end is guidable so that its leading end can be inserted into the proximal lateral aperture to subsequently emerge from the distal lateral aperture. This aspect is, accordingly, applied to a reamed nail, but also to an unreamed nail when the cavity is not through the entire length of the rod.

The means for guiding the elongate wire may comprise a guide wall or a guide channel mounted within the cavity, or more specifically and practically, a guide channel which includes a deflected guide wall portion.

Alternatively, the means for guiding the elongate wire may comprise a curving conduit mounted within the cavity and connecting between the proximal and distal lateral apertures.

To enable use of a guide wire as in the prior art, any additional bore or conduit as necessary, extending from the first end to the second end of the nail, or completing a passage through from one end to the other, may be provided in conjunction with any of the foregoing variants.

In many practical embodiments there will be two or more proximal lateral apertures and two or more distal apertures and appropriate guide provision will be made for guiding respective elongate wires into each proximal lateral aperture to emerge from respective distal lateral apertures.

According to a second aspect of the invention, an intramedullary nail is provided which need not have a central cavity, but nevertheless has curving conduit means extending between and connecting a proximal lateral aperture and a distal lateral aperture so as to guide the leading end of a flexible wire through from the proximal to the distal aperture. Thus, this aspect may be applied to an unreamed nail when the conduit is formed through the body of the nail itself. However, it may also be applied to a reamed nail where a separate conduit in the form of a central bore is provided for a guide wire, or where individual conduits, for example in the form of guide tubes, are provided in the cavity of a reamed nail. The latter arrangement also falls within the first aspect of the invention as defined above.

Nails according to the invention provide significant advantages over known nails in that their use allows for a quicker and yet highly reliable surgical procedure. A nail of the invention is insertable into the medullary cavity of a bone in a conventional manner. By use of a jig attached to the proximal end of the nail, a leading end of a flexible elongate wire which carries a cutting or grinding tip is accurately guided to the outside of the bone overlying one of the proximal lateral apertures of the nail, and is used to produce a hole in the bone at that position. Alternatively, to prevent blunting of the tip of this wire, the hole drilled through the bone adjacent to the proximal lateral aperture in the nail may be made with a drill guided by a jig. The wire is then inserted through the aperture and passed down the cavity, guided by the relevant guide means provided therein, or else is passed down a specific conduit, and guided out of the respective distal lateral aperture. The wire may then be rotated or reciprocated to engage the cutting or grinding tip with the bone tissue and create a hole in the bone adjacent the distal lateral aperture so that it can pass through and secure the distal part of the nail in the distal region of the bone. This is achieved without significantly disturbing the flesh covering the distal regional of the bone.

Thus, both ends of the nail can be secured to the bone in a reliable manner by use of only one or two fixing wires (although more may be possible) without any need for a second operation of fixation of the proximal or distal end by a transverse screw or bolt or other securing device. However, such fixation may still be undertaken if deemed necessary, and the fixing wires used in respect of the nail of the invention will not interfere with that.

The proximal and distal apertures may be round or elliptical and may be extended in the direction of the longitudinal axis of the nail.

### BRIEF DESCRIPTION OF DRAWINGS

The invention will now be described, by way of example, with reference to the accompanying drawings, in which:

FIG. 1 is a schematic perspective view, partially cut away, of a first practical embodiment of an intramedullary nail according to the first aspect of the invention with fixing wires extending therethrough as in use;

FIG. 2 is a cross section along II-II in FIG. 1;

FIG. 3 is a cross section along III-III in FIG. 1;

FIG. 4 is a schematic perspective view, again partially cut away, of a second practical embodiment of a nail according to the first aspect of the invention with fixing wires extending therethrough as in use;

FIG. 5 is a cross section along V-V in FIG. 4;

FIG. 6 is a cross section along VI-VI in FIG. 4;

FIG. 7 is a cross section at a position of insertion of fixing wires of a third embodiment of a nail according to the first aspect of the invention;

FIG. 8 is a comparable cross section at a central position along the third embodiment;

FIG. 9 is a comparable cross section at a position of exit of the fixing wires in the same, third embodiment;

FIG. 10 is a reduced scale side elevation of the dividing wall in the third embodiment;

FIG. 11 is a schematic side view of a conventional reamed nail, but showing internal detail;

FIGS. 12 to 15 are similar views illustrating various possibilities for application of the second aspect of the invention to reamed and unreamed nails;

FIG. 16 is a diagrammatic, exploded perspective view, to an enlarged scale, of a modified embodiment of a nail in accordance with the invention similar to that shown in FIG. 9;

FIG. 17 is a diagrammatic perspective view of part of a further embodiment of a nail in accordance with the invention;

FIG. 18 is a diagrammatic partially sectional perspective view of a fourth embodiment of a nail according to the first aspect of the invention; and

FIG. 19 is an enlarged scale partial view of the same nail rotated to show greater detail.

### DETAILED DESCRIPTION OF ILLUSTRATED AND OTHER EMBODIMENTS

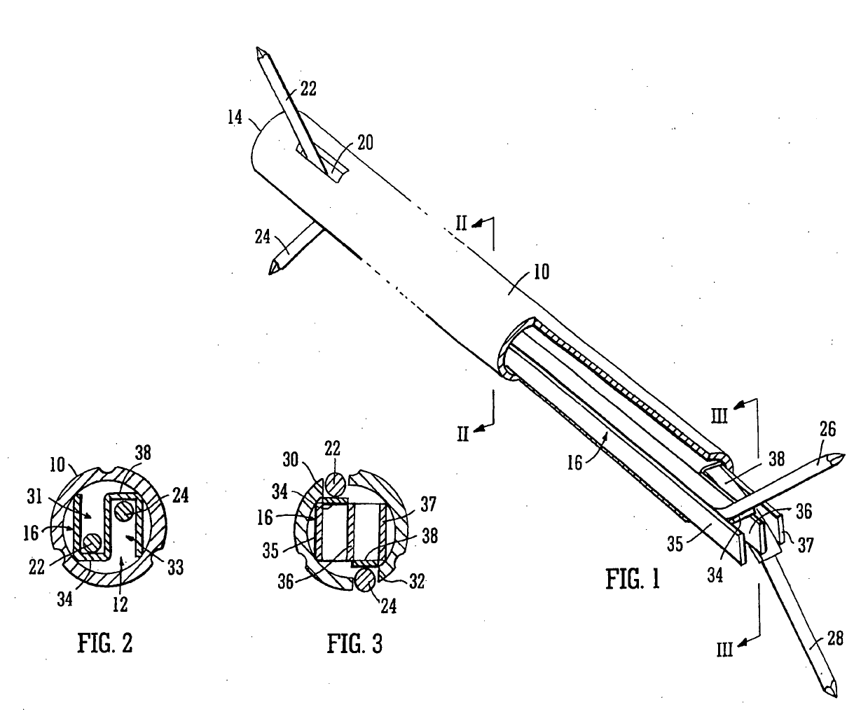

Referring firstly to FIGS. 1 to 3, a first practical embodiment of an intramedullary nail of the invention comprises an elongate rod 10 of stainless steel or titanium having a cavity 12 therethrough. Thus, it is hollow throughout. It has a first end 14, which may be termed a proximal end, and a second end, which may be referred to as a tip or distal end, although the latter is not shown in the drawings owing to the portion cut away to show the interior of the cavity 12. The rod 10 may taper from its proximal end to its tip, although again that is not shown in the drawings.

Two apertures, termed proximal lateral apertures, are formed in the wall of the rod 10 at a spacing from the proximal end 14. These are at substantially the same axial position in the rod, but a opposing positions circumferentially, and only one of these apertures 20 is apparent in FIG. 1.

Two generally corresponding apertures 30, 32 termed distal lateral apertures, are formed in the wall of the rod 10 at a spacing from the distal end or tip. These are positioned similarly to the proximal lateral apertures, ie at the same axial position, and opposite each other circumferentially, as is apparent in FIG. 3.

A channel element 16 is mounted inside the cavity 12, the purpose of which is to partition the cavity 12 and guide two elongate fixing wires 22, 24 therethrough. One wire, 22 in this case, is inserted into the proximal lateral aperture 20 at one side of the rod 10 and is guided through so that its leading end 26 emerges from the distal lateral aperture 30 at the same side, ie at substantially the same circumferential position, while the other wire 24 is inserted into the proximal lateral aperture (not shown) at the other side and is guided through so that its leading end 28 emerges from the distal lateral 32 also at that other side (again emerging from substantially the same circumferential position as where it was inserted). The trailing ends of the wires 22, 24 in each case remain projecting from the proximal lateral apertures, as shown in FIG. 1.

Thus, in use, after the nail has been inserted into the medulla of a bone in conventional manner as already explained in the introduction thereto, if the leading ends 26, 28 of the wires 22, 24 carry cutting or grinding tips, and the trailing ends are attached in turn to a drilling tool, they can be used, to cut through the bone overlying the proximal lateral apertures 20, then, after being guided through the nail, to cut through the bone overlying the distal lateral apertures 30, 32 and project therethrough. With both ends of the wire 22 or 24 then projecting the nail is secured to the bone adjacent each of its ends and at either side of the fracture. With two such wires 22 and 24 securing the nail at respective sides, the nail is more reliably retained. If threads are present at the leading end, these may grip the bone more securely.

The configuration of the channel element 16 to achieve this function of guiding both wires 22, 24 is apparent by reference to FIGS. 1 to 3. The element 16 has an angular S shaped profile, formed by bending a strip of bioacceptable metal, most suitably stainless steel or titanium. Thus, it provides two guide channels 31, 33 which are side by side but open in opposing directions, as shown in FIG. 2. The guide channels 31, 33 are defined by a common central wall 36 and respective base walls 34, 38, which together effectively divide the cavity 12 into two compartments, and also respective outer side walls 35, 37. The base walls 34, 38 of the respective channels 31, 33 serve as guide walls for guiding the path of the respective nails 22, 24.

In a region adjacent the distal lateral apertures 30, 32 the respective base walls 34, 38 are separated from the central and side walls 35, 36, 37 by longitudinal cuts along the bends in the channel element 16. In this region where they are separated, the base walls 34, 38 are deflected by definite bends or by gentle curves towards the open sides of their respective channels. The deflected regions of the base walls may be secured by spot welding to the side walls 35, 37 but this may not be necessary. They may reliably hold their deflected positions by the inherent inelasticity of the metal, particularly once the channel element 16 is mounted inside the hollow rod 10. The element 16 itself may be fixed in the cavity 12 by some spot welds, but again this may not be necessary.

In use, the wires 22, 24 are inserted into the proximal lateral apertures 20, as shown in FIG. 1 at an inclination to the surface of the rod 10. They pass into the respective channels 31, 33 and lie adjacent and are guided by the base walls 34, 38 as indicated in FIG. 2. As the wire tips 26, 28 approach the distal lateral apertures 30, 32 the deflected portions of the base walls 34, 38 guide them up from the channels 31, 33 to pass out through said apertures 30, 32 also at an inclination to the surface of the rod 10.

In the region of the channel element 16 adjacent the proximal lateral apertures 20, the base walls 34, 38 may also be deflected in a similar manner to that just described. However, that is probably not necessary as the wires 22, 24 will automatically pass into the respective channels 31, 33 at the point of insertion and so long as they are inserted at an inclination they will readily be guided down the cavity 12 upon contact with the base walls 34, 38.

Many other modifications in detail are possible as regards the embodiment which is shown schematically in FIGS. 1 to 3. For example in cases where the proximal or distal lateral apertures are at different axial positions along the rod, the channel element may be shorter or longer in respect of one of the channels by cutting away one of the side walls and base walls. The base walls will be separated and deflected in appropriate end regions for guidance of the fixing wires to and out of the distal lateral apertures, whatever their position. In cases where the proximal and distal lateral apertures are at different circumferential positions to each other the channel element may be twisted, at least for part of its length. Additionally, or alternatively, a side wall may have a separated and deflected region to serve a guide function or else a side wall may be cut away and the common central wall may have a separated and deflected region to serve a guiding function.

In other embodiments, the S-shaped channel element may be replaced by one of Z-shaped profile, ie lacking the side walls, or having V shaped channels or by appropriately conjoined or separate individual channel section elements. However, an S or Z profile is preferred for simplicity of production and assembly.

In other embodiments channel forming elements may be dispensed with and replaced by guide walls alone. The paramount consideration in respect of any device used in a surgical procedure is the ease of location within the cavity of the rod and reliability in guiding the wires.

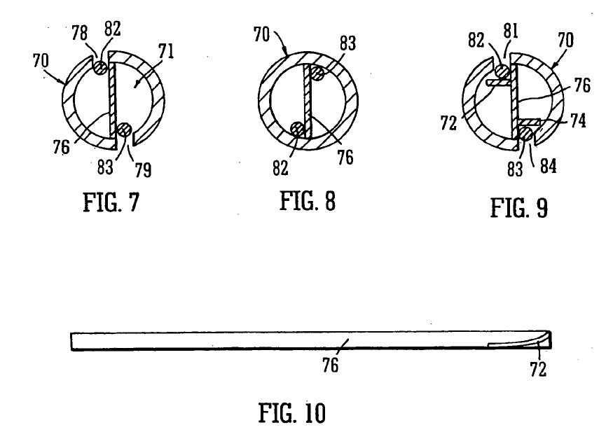

FIGS. 7 to 10 show such a simplified embodiment in which guide walls 72, 74 alone are provided, mounted onto an elongate dividing wall or partition 76. The nail 70 has its internal cavity 71 divided into two by the partition wall 76 which extends across the diameter of the cavity 71 and also from a position proximally of proximal lateral apertures 78, 79 to distally of distal lateral apertures 81, 84. The guide walls 72, 74 are relatively short and curving and are fixed to opposite sides of the partition 76 adjacent its distal end in the manner of ledges. Only one of the guide walls 72 is visible in FIG. 10. The other guide wall 74, which projects from the other side, curves in the opposite direction, so that the guide walls 72, 74 guide the wires 82, 83 out of distal lateral apertures 81, 84 at opposite sides of the nail 70.

Thus, as is apparent in FIGS. 7 and 9, one proximal lateral aperture 78 and one distal lateral aperture 81 lie at the same side of the partition 76 as each other and the guide wall 72 serves to guide the wire 82, after insertion into the proximal aperture 78, out of the corresponding distal aperture 81, which is located at a similar circumferential position on the nail 70. Similarly, both the other apertures 79, 84 lie at the other side of the partition 76 and the other guide wall 74 serves to guide the wire 83, after insertion into aperture 79, out of the other distal aperture 84.

Obviously in modified embodiments the guide walls could be differently mounted upon the partition to match the position of the respective distal apertures.

In manufacture, the guide walls are affixed to the sides of the partition, eg, by welding, and as a unit this is then located inside the nail cavity 71 and may be a friction fit or be secured by spot welds, for example.

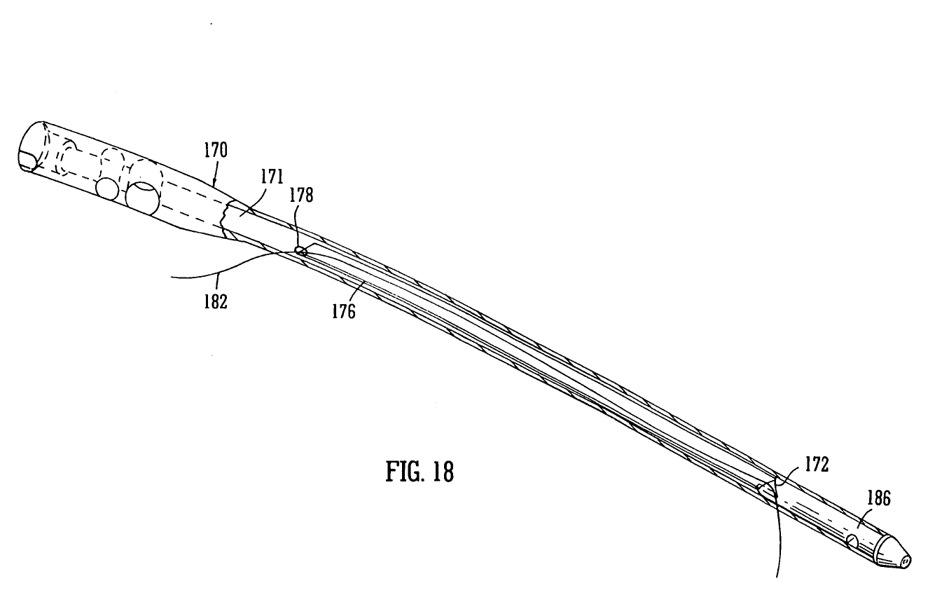

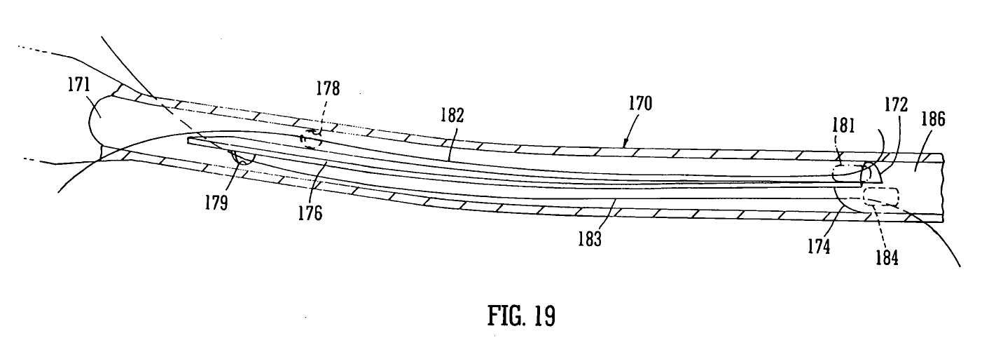

FIGS. 18 and 19 show another embodiment of a nail 170 in which guide walls 172, 174 in the manner of ledges are provided on an elongate partitioning wall 176. The nail 170 has its internal cavity 171 divided into two side-by-side channels by the partition wall 176 which extends across the diameter of the cavity 171 from a position adjacent proximal lateral apertures 178, 179 to a position adjacent distal lateral apertures 181, 184. In this embodiment, the partition wall 176 extends from a terminal enlargement 186 which serves as a plug to fix the partition wall 176 in the cavity 171. The guide walls 172, 174 are formed as shoulders or ledges where this enlargement 186 adjoins the partition wall 176. These shoulders are sloping in that each of them extends obliquely, at about 45° relative to the axis of the nail 170 (also relative to the plane of the wall 176). The respective guide wall shoulders 172, 174 slope in opposite directions at the respective sides of the partition wall 176.

Thus, as is apparent in FIGS. 18 and 19, one proximal lateral aperture 178 and one distal lateral aperture 181 lie at the same side of the partition 176 as each other and the guide wall shoulder 172 serves to guide one wire 182, after insertion into the proximal aperture 178, out of the corresponding distal aperture 181, which is located at a similar circumferential position on the nail 170. Similarly, both the other apertures 179, 184 lie at the other side of the partition wall 176 and the other guide wall shoulder 174 serves to guide another wire 183, after insertion into aperture 179, out of the other distal aperture 184.

Obviously in modified embodiments the guide walls could be differently oriented relative to the partition wall to match any differing position of the respective distal apertures.

Channel elements of alternate form to those described above or combinations of channel elements and guiding walls may be envisaged which would allow for insertion and guidance of more than two fixing wires, or for insertion of wires from separate proximal lateral apertures to emerge from the same enlarged or elongated distal lateral aperture or vice versa, if these were considered to be useful practical variants in orthopaedic surgery.

To facilitate the use of a central guide wire in introducing a nail in accordance with the above described embodiment, or any modifications thereof, into the medulla of a bone, conjoined channel section elements may be used to form the S-shaped profile, with a small central passageway being provided between the adjoining walls. A comparable arrangement is possible using two V-shaped elements to form a Z-shaped profile.

Although it is not apparent in the drawings, the distal region of the rod 10 or the nail 70 may be bent out of axial alignment with the remainder of the rod, as is conventional in respect of intramedullary nails designed for use in the medullary cavity of certain bones, such as the tibia.

In the case of intramedullary nails for use in treating fractures of the long bones, ie the femur and the humerus, there are technical/surgical problems involved in introducing a fixing wire in the groin area or in the area below an armpit. Accordingly, it is desirable in these circumstances, in accordance with the principles of the invention, to be able to insert nails into respective proximal lateral apertures at the same side of the nail (ie at approximately the same circumferential position, but different axial positions) to emerge from distal lateral apertures, as previously, at opposing sides of the nail.

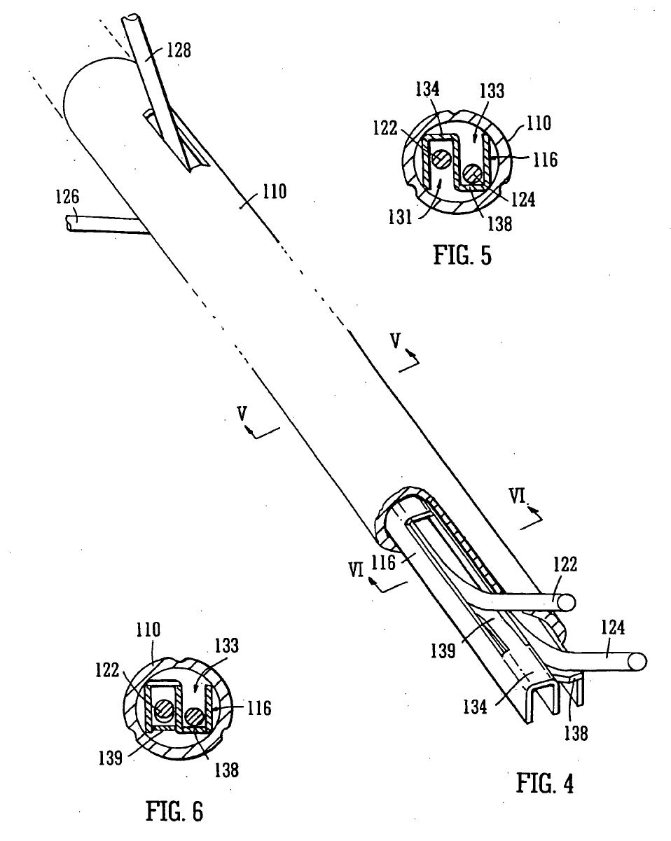

One way in which this can be accomplished by a modification to the basic embodiment illustrated in FIGS. 1 to 3, is shown in FIG. 4 to 6. In this case the hollow rod 110 is viewed in FIG. 4 with the wires 122 and 124 being inserted into proximal lateral apertures at the lower end of the drawing. An S-shaped channel element 116 providing two side by side guide channels 131, 133 open in opposite directions, as previously, is mounted in the cavity of the rod 110 and the leading ends 126, 128 of the wires 122, 124 are guided out by deflected base wall portions as previously. However, in order to guide the wire 122 into the cavity from the same side as the wire 124, a portion of the base wall 134 is cut as a flap 139 in the vicinity of the relevant proximal lateral apertures. This flap 139 has a free end directed towards the distal end of the rod 110 and it is deflected into channel to guide the wire into it in the distal direction as is apparent in FIGS. 4 and 6.

The other wire 124 is inserted into its proximal lateral aperture at a short axial and circumferential spacing from the first wire 122, and is at first guided by a terminally deflected portion of the base wall 138 of its channel, in the same manner already described in relation to FIGS. 1 to 3.

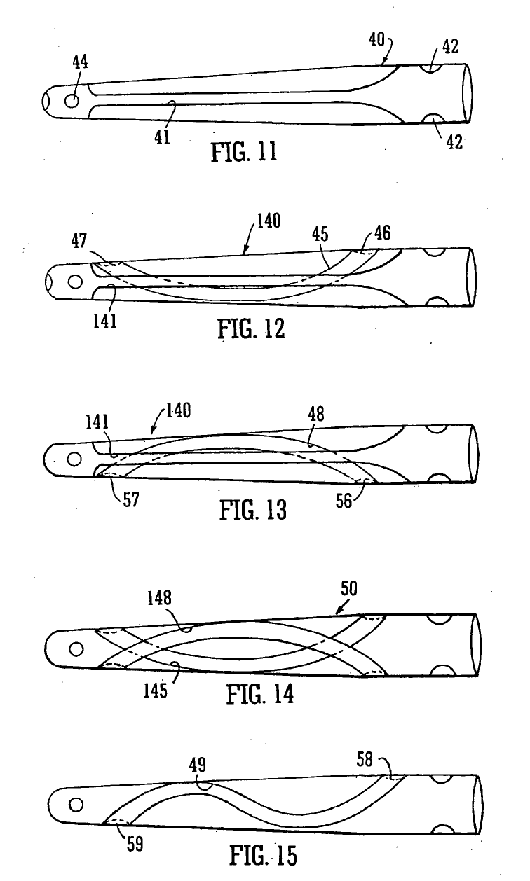

FIG. 11 illustrates a known type of reamed intramedullary nail 40, which has a narrow axial passageway 41 through its main central region to enable location over a guide wire (not shown), and wider hollow regions at each end in which opposed pairs of proximal 42 and distal 44 lateral apertures are formed, for insertion of transverse fixing devices, such as screws or bolts, as in the prior art.

FIG. 12 illustrates how, in accordance with a further embodiment of the invention, quite different to those illustrated in FIGS. 1 to 10, 18 and 19, an additional curving passageway 45 is formed through the material of the nail 140 from an additional proximal opening 46 to an additional distal opening 47. This passageway 45 is a conduit for guidance of a fixing wire (not shown) in the manner already explained in respect of FIGS. 1 to 3 above.

In FIG. 12, the passageway 45 is shown extending below the longitudinal plane of the axial passageway 141. In FIG. 13, a further such passageway 48 is shown of opposite curvature, extending between proximal and distal openings 56, 57 at the other side of the nail 140, circumferentially remote from the openings 46, 47. This passageway 48 is shown as being formed above the longitudinal plane of the axial passageway 141. Both such passageways 45, 48 may be provided in combination in either a reamed nail 140 (as in FIG. 11) or an unreamed nail 50, as shown in FIG. 14.

Nails as shown in FIGS. 12 to 14 may be manufactured as three longitudinal sections which have matching grooves on facing surfaces, which join together to provide the respective passageways 45, 48 or 145, 148 for guiding the fixing wires.

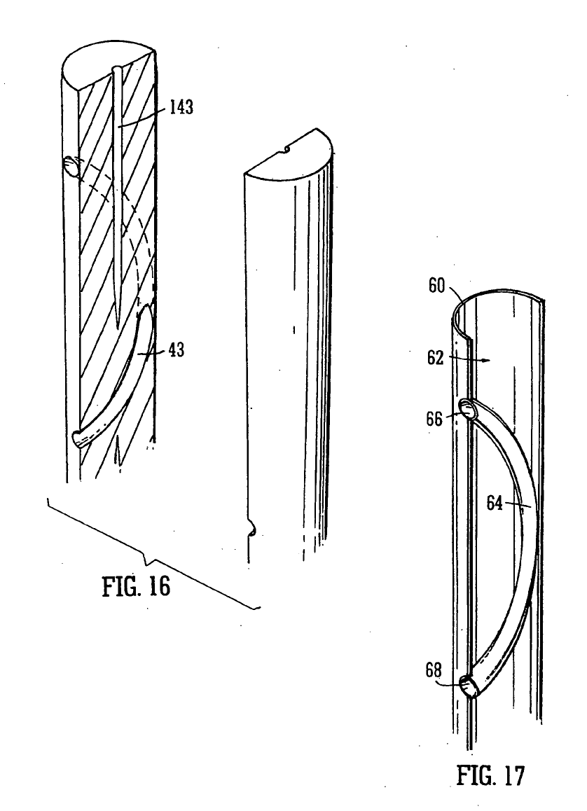

FIG. 15 shows a variation in which a doubly curving or S-shaped passage 49 is provided to guide a fixing wire from insertion into a proximal opening 58 at one circumferential position to emerge at a distal opening 59 at a remote substantially opposing circumferential position. Such a passageway 49 can be combined with a C-shaped curving passageway, as in FIG. 12 or FIG. 13, in either a reamed (including axial passageway) nail or an unreamed nail to allow insertion of two wires at the same side to emerge at opposing sides as is desirable for use in long bones, as discussed above.

FIG. 16 shows a modification, similar to FIG. 12 or 13, in which a similar curved passageway 43 is formed in a reamed nail, but may extend around from below to above the axial place in which an axial passageway 143 for a guide wire is disposed.

FIG. 17 shows a further embodiment in accordance with the invention in which a nail 60 having a large central cavity 62 has a curving tubular duct 64 mounted in that cavity and connecting between a proximal lateral aperture and a 66 at a distal lateral aperture 68 formed in the wall of the nail 60. The tubular duct 64 obviously provides a conduit which will guide the leading end or tip of a fixing wire (not shown) inserted into the proximal aperture 66 through and out of the distal aperture 68.

Modifications to the version shown in FIG. 17 may be provided which are similar to those explained in respect of FIGS. 12 to 15. In this respect two such tubes may be provided in an arrangement similar to that shown in FIG. 10, or a tube constrained to an S-shape may be provided, similar to FIG. 15, which may then be combined with a C-shaped curving tube.

Additional proximal and distal transverse securing screws or bolts, as in the prior art, may be used in conjunction with any of the foregoing embodiments, but the need for same can be assessed by the surgeon. In many cases it is believed this additional fixing will not be needed, which will reduce operating time and tissue disruption.

Many other modifications in detail are possible in respect of all the embodiments which have been described as will be apparent to one skilled in the art. The foregoing is to be considered only illustrative, not limitative of the scope of the invention.

1. An intramedullary nail comprising an elongate rod having a first end, a second end and an outer surface, a proximal lateral aperture in a region of the outer surface in the vicinity of the first end, a distal lateral aperture in a region of the outer surface in the vicinity of the second end and a cavity through at least a portion of the rod, at least between the proximal lateral aperture and the distal lateral aperture, and means within the cavity whereby a flexible elongate wire having a leading end and a trailing end is guidable so that its leading end can be inserted into the proximal lateral aperture to subsequently emerge from the distal lateral apertures, said means for guiding the elongate wire comprising a partition wall mounted within the cavity and a guide wall in the manner of a ledge provided on the partition wall.

2. An intramedullary nail as set forth in claim 1 wherein the guide wall is provided only in a region of the partition wall adjacent to the distal lateral aperture.

3. An intramedullary nail as set forth in claim 2 wherein the guide wall provides a sloping guide surface.

4. An intramedullary nail as set forth in claim 2 wherein the guide wall provides a curving guide surface.

5. An intramedullary nail as set forth in claim 1 comprising two proximal lateral apertures and two distal lateral apertures and wherein said partition wall divides said cavity into two channels with a respective proximal lateral aperture and a respective distal lateral aperture communicating with a respective one of said two channels, wherein said partition wall has opposing surfaces and a guide wall in the manner of a ledge formed on each of said opposing surfaces so that a first said guide wall serves to guide a first flexible elongate wire so that its leading end can be inserted into a first one of the proximal lateral apertures to emerge from a first one of the distal lateral apertures and a second said guide wall serves to guide a second flexible elongate wire so that its leading end can be inserted into a second one of the proximal lateral apertures to emerge from a second one of the distal lateral apertures.

6. An intramedullary nail as set forth in claim 5 wherein the partition wall extends from a terminal enlargement which serves as a plug to fix the partition wall in the cavity and wherein the respective guide walls are formed as shoulders where the enlargement adjoins the partition wall.

7. An intramedullary nail as set forth in claim 5 wherein the respective guide walls slope in opposite directions at the opposite sides of the partition wall.

8. An intramedullary nail as set forth in claim 5 wherein the respective walls curve in opposite directions at the opposite sides of the partition wall.

9. An intramedullary nail comprising an elongate rod having a first end, a second end and an outer surface, a proximal lateral aperture in a region of the outer surface in the vicinity of the first end, a distal lateral aperture in a region of the outer surface in the vicinity of the second end and a cavity through at least a portion of the rod, at least between the proximal lateral aperture and the distal lateral aperture, and a guide element mounted within the cavity, said guide element providing a channel having a deflected guide wall portion formed by partially separating said portion by cuts in the material of the channel so as to provide a flap and bending said flap relative to the remainder of the channel, whereby a flexible elongate wire having a leading end and a trailing end is guidable so that its leading end can be inserted into the proximal lateral aperture to subsequently emerge from the distal lateral aperture.

10. An intramedullary nail as set forth in claim 9 wherein said guide element defines at least two guide channels, each of which has a respective deflected guide wall portion.

11. An intramedullary nail as set forth in claim 9 comprising two proximal lateral apertures and two distal lateral apertures and wherein said guide element defines two guide channels with a respective proximal lateral aperture and a respective distal lateral aperture communicating with a respective one of said two channels, and wherein each of said said guide channels has a respective deflected guide wall portion so that a first deflected guide wall serves to guide a first flexible elongate wire so that its leading end can be inserted into a first one of the proximal lateral apertures to emerge from a first one of the distal lateral apertures and a second deflected guide wall portion serves to guide a second flexible elongate wire so that its leading end can be inserted into a second one of the proximal lateral apertures to emerge from a second one of the distal lateral apertures.

12. An intramedullary nail comprising an elongate rod having a first end, a second end and an outer surface, a proximal lateral aperture in a region of the outer surface in the vicinity of the first end, a distal lateral aperture in a region of the outer surface in the vicinity of the second end, and a curving conduit extending between and connecting the proximal and distal lateral apertures whereby a flexible elongate wire having a leading end and a trailing end is guidable so that its leading end can be inserted into the proximal lateral aperture to subsequently emerge from the distal lateral aperture.

13. A nail as set forth in claim 12 comprising two or more proximal lateral apertures and two or more distal lateral apertures with each proximal lateral aperture being connected to only one distal lateral aperture by a respective curving conduit.

14. A nail as set forth in claim 12 wherein the proximal lateral aperture is formed at a similar circumferential position on the outer surface to the distal lateral aperture to which it is connected and the conduit extending therebetween is approximately arcuate.

15. A nail as set forth in claim 12 wherein the or at least one of the proximal lateral apertures is formed at a circumferential position on the outer surface remote from the distal lateral aperture to which it is connected and the conduit extending therebetween is approximately S shaped.
